(19) United States
(12) Patent Application Publication
(10) Pub. No.: US 2011/0313207 A1
(43) Pub. Date: Dec. 22, 2011

(54) PROCESS FOR DISTILLATIVE WORKUP OF A METHANOL/WATER MIXTURE AND PROCESS FOR PREPARING ALKALI METAL METHOXIDES

(76) Inventors: Gerd Kaibel, Lampertheim (DE); Tilman Sirch, Schifferstadt (DE); Raimund Felder, Neustadt (DE); Josef Guth, Freinsheim (DE)

(21) Appl. No.: 13/203,504

(22) PCT Filed: Feb. 17, 2010
(86) PCT No.: PCT/EP2010/051981

§ 371 Date: Aug. 26, 2011

(30) Foreign Application Priority Data
Feb. 26, 2009 (EP) 09153704.3

(51) Int. Cl. B01D 3/00; C07C 29/00

(52) U.S. Cl. 568/840; 203/18

(57) ABSTRACT

The invention relates to a process for distillative workup of a methanol/water mixture, in which a methanol/water mixture is added to a distillation column (1), an essentially methanol-comprising vapor stream is withdrawn at the top of the distillation column (1) and an essentially water-comprising bottom stream at the bottom of the distillation column (1), at least a portion of the essentially methanol-comprising vapor stream is compressed and the compressed vapor stream is added as heating vapor to an evaporator (11) in which at least a portion of the methanol/water mixture to be separated is evaporated. The invention further relates to a process for preparing alkali metal methoxides in a reaction column (31) by adding methanol and alkali metal hydroxide solution to the reaction column (31), withdrawing alkali metal methoxide dissolved in methanol at the lower end of the reaction column (31) and withdrawing a methanol/water mixture at the upper end of the reaction column (31), and working up the methanol/water mixture by the process for distillative workup.

The invention relates to a process for distillative workup of a methanol/water mixture. The invention further relates to a process for preparing alkali metal methoxides in a reaction column, by adding methanol and alkali metal hydroxide solution to the reaction column, and withdrawing alkali metal methoxide dissolved in methanol at the lower end of the reaction column and a methanol/water mixture at the upper end of the reaction column. The methanol/water mixture is worked up by the process for distillative workup.

A mixture comprising methanol and water is obtained, for example, in the preparation of alkali metal methoxides from aqueous alkali metal hydroxide solution, optionally admixed with methanol, and methanol. Such a process for preparing alkali metal methoxides is described, for example, in EP-A 1 242 345. In this case, the reaction is performed in a reaction column to which an aqueous alkali metal hydroxide solution stream optionally admixed with methanol is fed at the top, and vaporous methanol in the lower region. At the top of the reaction column, a water-comprising methanol stream escapes. This stream is worked up in a rectification column. The stream comprising methanol and water is fed to the distillation column in which the workup is effected as a side stream, preferably in vaporous form. In the distillation column, the methanol/water mixture is separated in to a stream comprising essentially methanol, which is drawn off at the top of the column, and a stream comprising essentially water, which is drawn off at the bottom of the column. In order to supply the energy needed for distillation, at least a portion of the essentially water-comprising stream drawn off at the bottom of the column is evaporated in an evaporator. To this end, the evaporator is typically heated with heating vapor. This, however, has the disadvantage that a large amount of energy has to be supplied to the process from the outside. Especially in the case of heating with heating vapor, the generation of the heating vapor is thus first necessary. The methanol obtained at the top of the distillation column is partly condensed in a condenser and recycled into the column; the remaining portion is passed into the reaction column as feed.

A further process for preparing alkali metal methoxides is known from EP-A 1 997 794. In this process too, the methanol/water mixture obtained in the reaction column is fed to a distillation column in which the mixture is separated into an essentially methanol-comprising top stream and an essentially water-comprising bottom stream. The heating at the bottom of the column is likewise effected by means of a conventionally heated evaporator.

It is an object of the present invention to provide a process for working up a methanol/water mixture, which can be operated with a reduced requirement for heating vapor to be supplied compared to the known processes.

The object is achieved by a process for distillative workup of a methanol/water mixture, which comprises the following steps:
(a) adding the methanol/water mixture to a distillation column,
(b) withdrawing an essentially methanol-comprising vapor stream at the top of the distillation column and an essentially water-comprising bottom stream at the bottom of the column,
(c) compressing at least a portion of the essentially methanol-comprising vapor stream,
(d) adding the compressed vapor stream as heating vapor to an evaporator in which at least a portion of the methanol/water mixture to be separated is evaporated.

The compression of at least a portion of the essentially methanol-comprising vapor stream and the addition of the compressed vapor stream as heating vapor to an evaporator, in which at least a portion of the methanol/water mixture to be separated is evaporated can greatly reduce or in some cases even entirely eliminate the amount of heating vapor required to operate the distillation column. Energy only has to be supplied to operate the compressor for compression of the vapor stream. Since at least some of the vapor stream releases energy for evaporation and not all of the vapor stream is withdrawn from the process, this simultaneously also reduces the amount of energy to be supplied from outside.

In the context of the present invention, “essentially methanol-comprising” means a proportion of methanol of at least 99% by weight, preferably of at least 99.9% by weight and especially of at least 99.99% by weight.

“Essentially water-comprising” means a proportion of water in the stream of at least 95% by weight, preferably of at least 99% by weight and especially of at least 99.99% by weight.

In the context of the present invention, the withdrawal of the vapor stream at the top of the distillation column means that the vapor stream is withdrawn as a top stream or as a side draw above the internals in the distillation column. The essentially water-comprising bottom stream is withdrawn at the bottom of the column, typically at the lower tray of the column, but the withdrawal can also be effected via a side draw in the bottom.

The distillation columns used may be any desired distillation column known to those skilled in the art. Distillation columns which are typically used have internals. Suitable internals are, for example, trays, unstructured packings or structured packings. The trays used are typically bubble-cap trays, sieve trays, valve trays, tunnel-cap trays or slotted trays. Unstructured packings are generally random packings. The random packings used are typically Raschig rings, Pall rings, Berl saddles or Intalox® saddles. Structured packings are sold, for example, under the Mellapak® trade name from Sulzer. In addition to the internals mentioned, further suitable internals are known to those skilled in the art and can likewise be used.

Preferred internals have a low specific pressure drop per theoretical plate. Structured packings and random packings have, for example, a significantly lower pressure drop per theoretical plate than trays. This has the advantage that the pressure drop in the distillation column remains at a minimum, and hence the mechanical output of the compressor and the temperature of the methanol/water mixture to be evaporated remains low.

When structured packings or unstructured packings are present in the column, they may be divided, or a continuous packing may be present. Typically, however, at least two packings are present, one packing above the feed point of the methanol/water mixture and one packing below the feed point of the methanol/water mixture. When an unstructured packing is used, for example, a random packing, the random packings typically lie on a suitable sieve tray or grid tray.

In a preferred embodiment, the evaporator which is heated with the compressed portion of the essentially methanol-comprising vapor stream is an intermediate evaporator. This means that the evaporator is arranged above the bottom of the distillation column. This has the advantage that the temperature of the methanol/water mixture to be evaporated, which is withdrawn from the distillation column and is conducted through the evaporator, has a lower evaporation temperature than the essentially water-comprising bottom stream withdrawn at the bottom of the column. To evaporate the methanol/water mixture in an intermediate evaporator, the vapor stream thus need not be compressed to as high a degree, which necessitates a smaller energy input for compression.

In addition to the heating of an intermediate evaporator with the compressed vapor stream, it is, however, also possible to heat an evaporator with which at least a portion of the bottom stream of the distillation column is heated. However, this requires greater compression of the vapor stream and hence a greater amount of energy required to compress the vapor stream.

The evaporator to which the compressed vapor stream is added as heating vapor is therefore preferably an intermediate evaporator.

The evaporator is typically arranged outside the distillation column. Via a draw from the column, the methanol/water mixture to be evaporated in the evaporator is drawn off and fed to the evaporator. Via a feed, the evaporated mixture, optionally with a residual proportion of liquid, is recycled back into the distillation column. When the evaporator is an intermediate evaporator, the draw through which the methanol/water mixture is drawn off and fed to the evaporator is a side draw, and the feed, through which the evaporated methanol/water mixture is fed back to the column is a side feed. When the evaporator heats the bottom of the column, at least a portion of the bottom draw stream is fed to the evaporator, evaporates in the evaporator and is recycled back into the column in the region of the bottom. Alternatively, it is, however, also possible to form tubes through which the compressed vapor stream flows, for example on a suitable tray in the case of use of an intermediate evaporator, or in the bottom of the column. In this case, the evaporation is effected on the tray or in the bottom of the column. It is preferred, however, to arrange the evaporator outside the column.

Suitable evaporators, which can be heated with the compressed vapor stream, are, for example, natural circulation evaporators, forced circulation evaporators, forced circulation flash evaporators, steam boilers, falling film evaporators or thin film evaporators. The heat transferer used for the evaporator is typically a tube bundle or plate apparatus in the case of natural circulation evaporators and forced circulation evaporators. In the case of use of a tube bundle transferer, either the compressed vapor stream can flow through the tubes and the methanol/water mixture to be evaporated around the tubes, or the compressed vapor stream flows around the tubes and the methanol/water mixture to be evaporated flows through the tubes. In the case of a falling film evaporator, the methanol/water mixture to be evaporated is typically added as a thin film on the inside of a tube and the tube is heated from the outside. In contrast to a falling film evaporator, in a thin film evaporator, a rotor with wipers is additionally provided, which distributes the liquid to be evaporated on the inner wall of the tube to a thin film.

In addition to those mentioned, however, it is also possible to use any other evaporator design which is known to those skilled in the art and is suitable for use on a distillation column.

When the evaporator, which is operated with the compressed vapor stream as heating vapor is an intermediate evaporator, it is preferred when the intermediate evaporator is arranged in the stripping section of the distillation column or in the region of the feed point of the methanol/water mixture. The arrangement of the intermediate evaporator in the stripping section of the distillation column or in the region of the feed point of the methanol/water mixture can allow a predominant portion of the heating energy to be introduced through the intermediate evaporator. For example, it is possible to introduce more than 80% of the energy through the intermediate evaporator. According to the invention, the intermediate evaporator is preferably arranged and/or configured such that it introduces more than 50%, especially more than 75%, of the total energy required for the distillation.

In the case of use of an intermediate evaporator, it is especially advantageous when the intermediate evaporator is arranged such that the distillation column has 1 to 50 theoretical plates below the intermediate evaporator and 1 to 200 theoretical plates above the intermediate evaporator. It is especially preferred when the distillation column has 2 to 10 theoretical plates below the intermediate evaporator and 20 to 50 theoretical plates above the intermediate evaporator.

The side draw stream through which the methanol/water mixture is fed to the intermediate evaporator, and the side feed through which the evaporated methanol/water mixture from the evaporator is fed back to the distillation column, may be positioned between the same trays of the distillation column. It is, however, also possible that side draw and side feed are at different heights.

In a preferred embodiment, in the case of use of an intermediate evaporator, the diameter of the distillation column above the intermediate evaporator is greater than the diameter of the distillation column below the intermediate evaporator. This has the advantage that capital costs can be saved.

The vapor stream can be compressed in step (c) in any desired manner known to those skilled in the art. For example, the compression can be carried out in one stage or more than one stage. In the case of multistage compression, it is possible to use a plurality of evaporators of the same design or evaporators of different design.

The use of a one-stage compression or of a multistage compression depends on the pressure to which the vapor stream is to be compressed. When the compressed vapor stream is used to heat an intermediate evaporator, the pressure difference to be overcome with the compressor is less than in the case of use of the vapor stream to heat a compressor at the bottom of the distillation column. The greater pressure difference to be overcome can be overcome by additional compressor stages or by a more powerful compressor. Typically, however, additional compressor stages are used.

Suitable compressors for compressing the vapor stream are any desired compressors known to those skilled in the art, with which gas streams can be compressed. Suitable compressors are, for example, one-stage or multistage turbines, piston compressors, screw compressors, centrifugal compressors or axial compressors.

In the case of multistage compression, compressors suitable for the pressure stages to be overcome in each case are used.

In addition to the evaporator operated with the compressed vapor stream, preference is given to using a further, conventionally heated evaporator arranged at the bottom of the distillation column. When the evaporator heated with the vapor stream is likewise arranged at the bottom of the column, the conventionally heated evaporator can be utilized, for example, to additionally supply heat during the operation of distillation columns. In general, the conventionally heated evaporator is, however, utilized to start up the column. On startup of the column, sufficient vapor with which the evaporator can be heated is not yet available, such that heat at first has to be supplied from the outside. After the startup of the distillation column, the amount of vapor which can be withdrawn at the top of the column increases, and the operation can be switched to the evaporator heated with the compressed vapor stream. In this context, it is firstly possible to slowly put the evaporator heated with the vapor stream into operation and to heat the conventionally heated evaporator to a correspondingly lesser degree, or establishment of a steady operating state in the distillation column is awaited, and then there is a switch from the conventionally heated evaporator to the evaporator heated with the compressed vapor stream.

When an intermediate evaporator is heated with the compressed vapor stream, the additional conventionally heated evaporator is utilized in order to introduce further heat into the distillation column at the bottom of the column. In this case, the conventionally heated evaporator is operated over the entire operating time of the distillation column. Here, too, for startup of the column, a greater amount of heat has to be introduced at first into the distillation column via the conventionally heated evaporator until the intermediate evaporator can be supplied with a sufficiently large vapor stream to heat it. Then the amount of heat which is introduced with the conventionally heated evaporator into the distillation column can be reduced. Alternatively, in the case of use of an intermediate evaporator, it is also possible to use two conventionally heated evaporators at the bottom of the column. The additional conventionally heated evaporator is then utilized for startup of the column, and the other conventionally heated evaporator is operated further during the operation of the column.

The methanol/water mixture to be separated in the distillation column can be supplied in liquid or gaseous form. Preference is given, however, to supplying the methanol/water mixture in gaseous form. The methanol/water mixture is preferably added via a side feed.

When the methanol/water mixture to be separated in the distillation column originates from the preparation of alkali metal methoxides, the methanol/water mixture is preferably added to the distillation column in gaseous form.

The pressure with which the distillation column is operated is preferably in the range from 0.2 to 10 bar, especially in the range from 0.5 to 3 bar.

In one embodiment of the invention, a portion of the essentially methanol-comprising vapor stream is condensed and passed back into the distillation column. The condensed portion of the vapor stream which is passed back into the distillation column can firstly be withdrawn from the compressed vapor stream with which the evaporator is heated, or alternatively from an additional portion which is not compressed. When a portion of the vapor stream with which the evaporator is operated is condensed, and recycled into the distillation column, the portion recycled into the distillation column is preferably decompressed to the operating pressure of the distillation column before being added to the distillation column. The recycling of the condensed vapor stream into the top of the column increases the concentration of methanol and achieves improved removal of methanol and a greater purity of the methanol in the vapor stream.

When a portion of the essentially methanol-comprising vapor stream is condensed and passed back into the distillation column, the reflux ratio is preferably at least 0.4, especially 0.8 to 1.4.

In order to prevent inert gases from accumulating, it is advantageous to connect a liquid separator downstream of the vapor compressor. Gases removed in the liquid separator can then be discharged from the process in substreams or completely, which also removes inert gases present.

When the process for distillative workup of the methanol/water mixture is used in a process for preparing alkali metal methoxides, the methanol/water mixture is typically withdrawn from a reaction column for preparing the alkali metal methoxides as a top stream. The methanol/water mixture withdrawn as the top stream can then be fed directly into the distillation column. Alternatively, it is, however, also possible to use a vapor compressor in which the methanol/water mixture withdrawn as the top stream is compressed and then fed to the distillation column. The compression of the methanol/water mixture further reduces the energy required for the distillation in the distillation column.

In addition to the process for distillative workup of the methanol/water mixture, the invention also relates to a process for preparing alkali metal methoxides in a reaction column, wherein methanol and alkali metal hydroxide solution are added to the reaction column. At the lower end of the reaction column, alkali metal methoxide dissolved in methanol is withdrawn. At the upper end of the reaction column, a methanol/water mixture is withdrawn, which is then worked up by distillation according to the above-described process for working up a methanol/water mixture.

The methanol used to prepare the alkali metal methoxide may, in the inventive configuration of the process, also be commercial methanol with a methanol content of more than 99.8% and a water content of up to 0.1%. The methanol can either be supplied in the rectifying section of the distillation column or directly at the top. The optimal feed point depends on the water content of the methanol used, and secondly on the desired residual water content in the distillate. The higher the proportion of water in the methanol used and the higher the purity requirement in the distillate, the more favorable is a feed a few theoretical plates below the top of the distillation column. Preference is given to up to 20 theoretical plates below the top of the distillation column and especially 1 to 5 theoretical plates.

The reaction column, in which the alkali metal methoxide is prepared comprises preferably at least 2, especially 15 to 40, theoretical plates between the feed point of the alkali metal hydroxide solution and the feed point of the methanol.

In general, the reaction column comprises internals. Suitable internals are, for example, trays, structured packings or random packings. When the reaction column comprises trays, bubble-cap trays, valve trays or sieve trays are suitable. When the reaction column comprises trays, preference is given to selecting those trays with which not more than 5%, preferably less than 1%, of the liquid trickles through the particular trays. The construction measures required to minimize trickle of the liquid are familiar to those skilled in the art. In the case of valve trays, for example particularly tightly sealing valve designs are selected. Reducing the number of valves additionally allows the vapor velocity in the tray orifices to be increased to twice the value which is typically established. In the case of use of sieve trays, it is particularly favorable to reduce the diameter of the tray orifices and to maintain or even increase the number of orifices.

In the case of use of structured or unstructured packings, structured packings are preferred with regard to the homogeneous distribution of the liquid. In this embodiment, in addition, in any parts of the column cross section which correspond to more than 2% of the total column cross section, the averaged value of liquid to vapor stream with regard to the liquid must not be exceeded by more than 15%, preferably by not more than 3%. Keeping this amount of liquid low makes it possible that the capillary effect at the wire meshes rules out local peak values in the liquid trickle density.

In the case of columns with unstructured packings, especially with random packings, and in the case of columns with structured packings, the desired characteristics of the liquid distribution can be achieved by, in the edge region of the column cross section adjacent to the column jacket, which corresponds to about 2 to 5% of the total column cross section, reducing the liquid trickle density compared to the remaining cross-sectional areas up to 100%, preferably by 5 to 15%. This can be achieved, for example, by the controlled distribution of the drip-off sites of the liquid distributors or the bores thereof with simple means.

The alkali metal methoxide can be prepared either continuously or batchwise. Preference is given to continuous preparation. In continuous processes, the alkali metal hydroxide solution, typically in aqueous form, optionally admixed with methanol, is fed in at the top of the reaction column. The reaction column is operated as a pure stripping column. In the lower region of the column, methanol is fed in vaporous form. On-spec alkali metal methoxide is obtained via the bottom draw. The methanol stream which still comprises water and leaves at the top of the column, referred to above as methanol/water mixture, is worked up by distillation as described above. The methanol obtained in the distillation is then fed back to the reaction column.

The amount of methanol used is selected such that it serves simultaneously as a solvent for the alkali metal methoxide obtained. The amount of the methanol is preferably selected such that the desired concentration of the alkali metal methoxide solution is present in the bottom of the reaction column.

Alkali metal hydroxide solutions used customarily are sodium hydroxide solution and potassium hydroxide solution.

The reaction column is preferably operated with no return line. This means that all of the methanol/water mixture withdrawn in vaporous form is fed to the distillation column. The methanol/water mixture is preferably fed in vaporous form to the distillation column.

When a portion of the methanol is added in vaporous form at the upper end or in the region of the upper end of the reaction column, the dimensions in the lower region of the reaction column can be reduced. When a portion of the methanol is added in vaporous form at the upper end or in the region of the upper end of the reaction column, only a portion of 10 to 70%, preferably of 30 to 50%, is fed in at the lower end of the reaction column, and the remaining portion, in a single stream or distributed between a plurality of substreams, is added in vaporous form preferably 1 to 10 theoretical plates, more preferably 1 to 3 theoretical plates, below the feed point of the alkali metal hydroxide solution.

The arrangement of one or more intermediate evaporators in the upper region of the reaction column allows the dimensions in the lower region of the reaction column to be reduced. In the case of the embodiment with intermediate evaporators, it is also possible to feed in substreams of the methanol in liquid form in the upper region of the reaction column.

The reaction column is preferably operated at a pressure in the range from 0.5 to 40 bar, preferably in the range from 1 to 5 bar, more preferably in the range from 1 to 3 bar, since lower heating outputs and smaller amounts of methanol can be achieved at a higher pressure.

In the integrated system composed of reaction column and distillation column, in the case of preparation of alkali metal methoxides, the distillation column is preferably operated at a pressure which is selected such that the pressure gradient between the columns in the case of a vapor compression is possible with a low level of complexity for the methanol/water mixture or alternatively for the methanol stream fed to the reaction column.

The methanol required for the reaction and the dilution of the alkali metal methoxide solutions is added at the top of the distillation column at temperatures up to the boiling point, preferably at room temperature. In this case, a dedicated feed can be provided for the additional methanol, or else, in the case of recycling of a portion of the methanol withdrawn at the top of the distillation column, after the condensation, the additional methanol can be mixed with the latter and they can be fed together into the column. In this case, it is particularly preferred when the fresh methanol is added to a condensate vessel in which the methanol condensed out of the vapor stream is collected.

In an advantageous configuration of the invention, the reaction column and the distillation column for workup of the methanol/water mixture are accommodated in one column jacket, the lower region of the column being divided by a dividing wall. In this case, the reaction to give the alkali metal methoxide is performed in one part of the column, the alkali metal hydroxide solution being added at about the level of the upper end of the dividing wall and the methanol being introduced in vaporous form at the lower end. The methanol/water mixture which forms above the addition point of the alkali metal hydroxide solution is then distributed above the dividing wall over the entire column region which serves as the rectifying section of the distillation column. The second, lower part of the column delimited by the dividing wall is the stripping section of the distillation column. The energy needed for the distillation is then supplied via an evaporator at the lower end of the second part of the column delimited by the dividing wall, this evaporator being heatable conventionally or heatable with the compressed vapor stream. When the evaporator is heated conventionally, an intermediate evaporator heated with the compressed vapor stream is additionally provided.

Through the return line in the upper part of the column above the dividing wall, a portion of the methanol/water mixture to be separated also runs back into the part of the dividing wall column which serves as the reaction column. A suitable configuration of the tray directly above the part of the dividing wall column which serves as the reaction column, however, allows this proportion which runs back into the reaction column to be minimized.

Embodiments of the invention are shown in the drawings and are explained in detail in the description which follows.

The figures show:

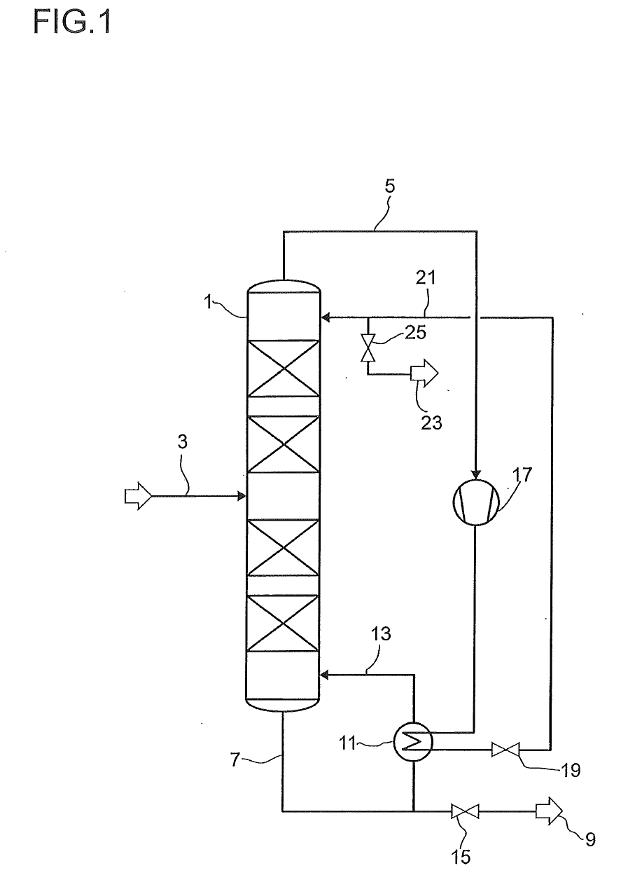

FIG. 1 a process flow diagram of the process according to the invention for distillative workup of a methanol/water mixture,

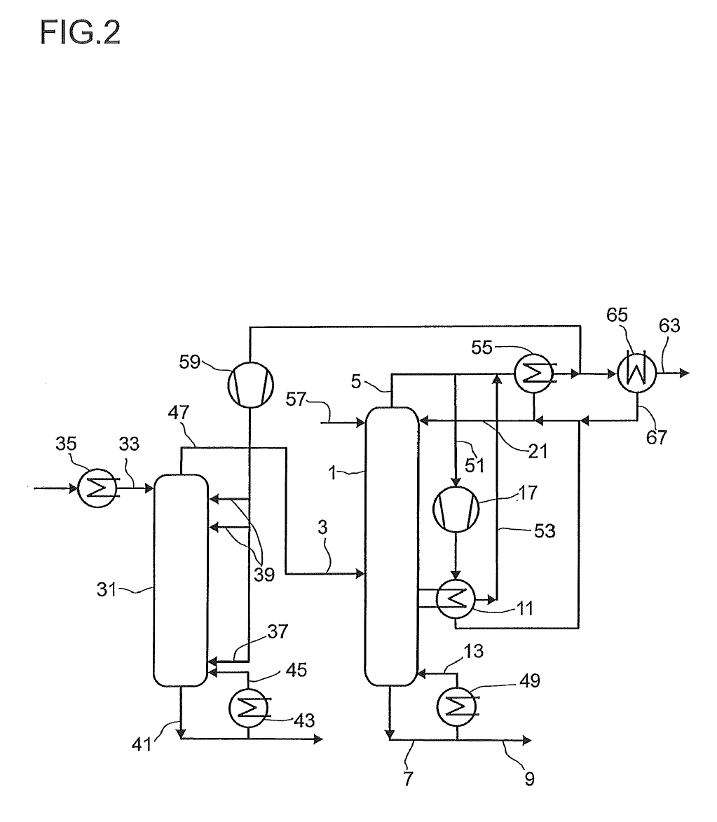

FIG. 2 a process flow diagram for a process for preparing alkali metal methoxide in a first embodiment,

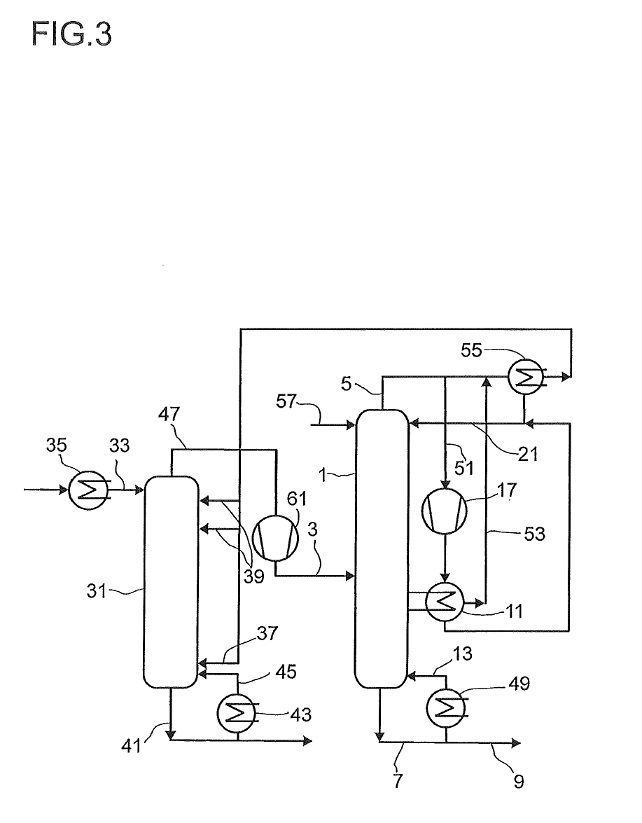

FIG. 3 a process flow diagram for a process for preparing alkali metal methoxide in a second embodiment,

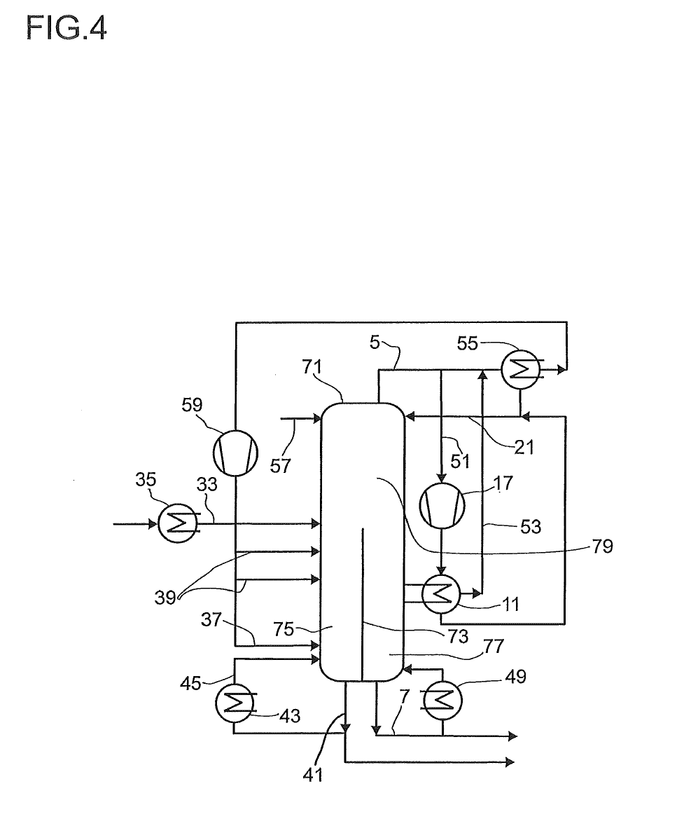

FIG. 4 a process flow diagram for a process for preparing alkali metal methoxide in a third embodiment.

FIG. 1 shows a process flow diagram for a process according to the invention for distillative workup of a methanol/water mixture.

A methanol/water mixture is fed via a feed 3 to a distillation column 1. The distillation column 1 may be equipped with trays, unstructured packings, especially random packings, or structured packings. When the distillation column 1 comprises packings, it is preferred to provide two packings, one packing above the feed 3 and one packing below the feed 3. It is also possible in each case for more than one packing to be provided above the feed 3 and below the feed 3.

The feed 3 is preferably a side feed and is preferably arranged such that 1 to 50, especially 2 to 10, theoretical plates are present below the feed 3, and 1 to 200, especially 20 to 50, theoretical plates above it.

In the distillation column 1, the methanol/water mixture is separated into an essentially methanol-comprising vapor stream and an essentially water-comprising bottom stream. The essentially methanol-comprising vapor stream is withdrawn via a vapor draw 5 at the top of the column. As in the embodiment shown here, the vapor draw 5 may be at the top of the distillation column 1, but it is also possible to configure the vapor draw 5, for example, as a side draw of the distillation column 1. The configuration of the vapor draw 5 as a side draw also allows the use of methanol, which possesses low-boiling fractions, for example alkanes such as butane. The low-boiling fractions can, in the case of a vapor draw 5 configured as a side draw, be discharged at the top of the distillation column 1 with a small loss of methanol. It is preferred, however, to position the vapor draw 5 at the top of the distillation column 1. In order not to discharge any liquid with the vapor from the distillation column 1, it is preferable to provide, below the vapor draw 5, a droplet separator which is present as an additional internal in the distillation column 1. The droplet separator used may, for example, be an additional packing element or any desired commercial droplet separator known to those skilled in the art.

Via a bottom outlet 7, the essentially water-comprising bottom stream is removed. A portion of the essentially water-comprising bottom stream is withdrawn from the process via an outlet 9. Another portion of the essentially water-comprising bottom stream is at least partly evaporated in an evaporator 11 and fed to the distillation column 1 at the lower end via a feed 13. The ratio of recycled stream and stream withdrawn via the outlet 9 can be adjusted by means of a valve 15.

According to the invention, the evaporator 11 is heated with the essentially methanol-comprising vapor stream withdrawn via the vapor draw 5. To this end, the essentially methanol-comprising vapor stream is compressed in a vapor compressor 17. The pressure to which the essentially methanol-comprising vapor stream is compressed in the vapor compressor 17 depends on the temperature required to evaporate the essentially water-comprising bottom stream 13. The vapor can be compressed in one stage in a vapor compressor 17 or in a plurality of stages by means of a plurality of vapor compressors connected in series. Multistage evaporation in one apparatus which has a plurality of compressor stages is also possible. By virtue of the release of heat for evaporation of the essentially water-comprising bottom stream in the evaporator 11, at least a portion of the vapor stream is condensed. In a throttle 19, the vapor stream is decompressed to the operating pressure of the distillation column 1 and recycled into the latter via a feed 21 at the top of the distillation column 1. A methanol draw 23 is used to withdraw a portion of the essentially methanol-comprising vapor stream from the process. The ratio of methanol-comprising vapor stream recycled into the distillation column 1 and essentially methanol-comprising vapor stream withdrawn from the process via the methanol draw 23 is adjusted by means of a valve 25. The ratio of recycled and withdrawn vapor stream depends on the desired purity of the methanol in the vapor stream.

FIG. 2 shows a process flow diagram for a process for preparing alkali metal methoxide in a first embodiment.

To prepare alkali metal methoxide, aqueous alkali metal hydroxide solution which has optionally been admixed with methanol or a methanol/water mixture is fed to a reaction column 31 via a feed 33. The alkali metal hydroxide solution can also be supplemented with methanol or a methanol/water mixture in a separate feed stream. The feed 33 accommodates a heat transferer 35 through which the aqueous alkali metal hydroxide solution is conducted before it flows into the reaction column 31. In the heat transferer 35, the aqueous alkali metal hydroxide solution is heated to the temperature of the feed point. Optionally, the aqueous alkali metal hydroxide solution is partly evaporated in the heat transferer 35. The feed 33 for addition of the aqueous alkali metal hydroxide solution, which is optionally admixed with methanol or a methanol/water mixture, is positioned at the upper end of the reaction column 31.

Via a methanol feed 37 positioned at the lower end of the reaction column 31, vaporous methanol is introduced into the reaction column 31. In addition to the feeding with the methanol feed 37 at the lower end of the column, further vaporous methanol can be fed in at the upper end of the reaction column via one or more further methanol feeds 39. The further methanol feeds 39 are preferably 1 to 10 theoretical plates, especially 1 to 3 theoretical plates, below the feed 33 for the aqueous alkali metal hydroxide solution.

Via a bottom outlet 41, the alkali metal methoxide prepared in the reaction column 31 is withdrawn. A portion of the alkali metal methoxide is recycled into the reaction column 31 via an evaporator 43 and a return line 45, in order thus to adjust the concentration of the alkali metal methoxide solution withdrawn at the bottom outlet 41.

At the top of the reaction column 31, a methanol/water mixture is withdrawn in vaporous form via a vapor draw 47.

For workup, the methanol/water mixture withdrawn at the top of the reaction column 31 is fed to the distillation column 1 via the feed 3. In the distillation column 1, the methanol/water mixture is separated into an essentially water-comprising bottom stream and an essentially methanol-comprising top stream.

Via the bottom outlet 7, the essentially water-comprising bottom stream is withdrawn from the distillation column 1. A portion of the essentially water-comprising bottom stream is at least partly evaporated in the evaporator 49 and recycled into the distillation column 1 via the feed 13. The portion of the essentially water-comprising bottom stream which has not been recycled into the distillation column 1 is withdrawn via the outlet 9.

In the embodiment shown here, the evaporator 49, in which the essentially water-comprising bottom stream is at least partly evaporated, is a conventionally heated evaporator. The evaporator 49 is preferably heated with steam. However, any other way of heating the evaporator 49 known to those skilled in the art is also conceivable.

The essentially methanol-comprising vapor stream is withdrawn via the vapor draw 5 at the top of the distillation column 1. From the vapor draw 5, a line 51 branches off, in which the vapor compressor 17 is positioned. In the vapor compressor 17, the essentially methanol-comprising vapor stream is condensed in order to heat the evaporator 11. In the embodiment shown here, the evaporator 11 is an intermediate evaporator, in which a portion of the methanol/water mixture to be separated in the distillation column 1 is evaporated. To this end, for example, a portion of the methanol/water mixture to be separated is withdrawn via a side draw on the distillation column 1, passed through the evaporator and recycled into the distillation column. The draw and the feed through which the methanol/water mixture to be evaporated is passed, may be at the level of the same theoretical plate or else at different positions in the distillation column 1. When feed and outlet are at different positions, it is preferred when the draw through which the evaporator 11 is supplied with the methanol/water mixture is above the feed in the distillation column 1 through which the evaporated methanol/water mixture is recycled into the distillation column 1.

The evaporator 11 which, as shown here, is utilized as an intermediate evaporator is preferably positioned such that 2 to 10 theoretical plates of the distillation column 1 are below the evaporator 11 and 20 to 50 theoretical plates are above the evaporator.

In the evaporator 11, the essentially methanol-comprising vapor stream is cooled by the heat transfer to the methanol/water mixture to be evaporated and may be partly or even fully condensed. The cooled, optionally at least partly condensed methanol stream is then recycled via the feed 21 at least partly back into the distillation column 1. The portion of the essentially methanol-comprising vapor stream which has not been condensed out in the evaporator 11 is recycled back into the vapor draw 5 via a vapor line 53 beyond the point at which the line 51 branches off. The vapor draw 5 then opens into a condenser 55. Alternatively, it is also possible that the vapor line 53 opens into the condenser 55 in addition to the vapor draw 5 and separately therefrom. In the condenser 55, a portion of the essentially methanol-comprising vapor stream is condensed and recycled via the feed 21 into the distillation column 1. In this case, the portion of the essentially methanol-comprising vapor stream condensed out in the condenser 55 can first open into the feed 21 originating from the evaporator 11, and all of the condensed methanol can be recycled together into the distillation column 1, or else the feed 21 originating from the evaporator 11 opens into the distillation column 1 independently of the methanol stream originating from the condenser 55. The proportion of the essentially methanol-comprising vapor stream which has not been condensed in the condenser 55 is recycled via the methanol feed 37 and optionally the at least one further methanol feed 39 into the reaction column 31. Methanol consumed in the reaction column 31 is added via a methanol feed 57 at the top of the distillation column 1. The addition of the methanol via the methanol feed 57 at the top of the distillation column can further increase the purity of the essentially methanol-comprising vapor stream drawn off via the vapor draw 5. In this way, the proportion of water introduced into the reaction column 31 with the methanol is reduced further. As well as the addition of the methanol via the methanol feed 57 at the top of the column, it is also possible to arrange the methanol feed 57 up to 10 theoretical plates below the top of the distillation column 1.

A portion of the vapor leaving the condenser 55 can be discharged. This allows, for example, inerts which are entrained into the process to be discharged. The inerts get into the process, for example, in the form of gases dissolved in methanol and/or alkali. It is also possible for small amounts of barrier gas to get into the process through seals. For discharge of the inerts, in the embodiment shown here, downstream of the condenser 55, a line 63 branches off, through which a portion of the uncondensed vapor stream is withdrawn. In a further condenser 65, the vapor stream is cooled further. The cooling medium used in the condenser 65 is, for example, water or brine. A preferred cooling medium is brine. The use of brine makes it possible to achieve a lower temperature than with water, such that a greater proportion of the vapor can condense. The proportion uncondensed in the condenser 65, which comprises the gaseous inerts, is withdrawn from the process and can be sent, for example, to a flare or to an offgas scrubber for further treatment. The condensed proportion is recycled into the distillation column via a return line 67. The return line 67 can, as shown here, open into the feed 21, or else be configured as a separate feed into the distillation column 1.

In the case of use of the further condenser 65, the condenser 55 can be dispensed with in one embodiment. In this case, the return line necessary to achieve the desired purity of the methanol is implemented via the condenser 65. The condenser 65 may have either a one-stage or multistage configuration, preferably a three-stage configuration. In this case, cooling is effected in one stage with air, in a further stage with water and in the third stage with brine.

In an embodiment which is not shown here, it is also possible to use the proportion of the essentially methanol-comprising vapor stream which has been condensed out in the evaporator 11 to further heat the vaporous proportion of the essentially methanol-comprising vapor stream drawn off from the condenser 55 before it is fed into the reaction column 31 via the methanol feeds 37, 39.

The vapor stream which has been drawn off via the vapor draw 5 of the distillation column 1 and fed via the methanol feed 37 and the further methanol feeds 39 into the reaction column 31 can be compressed in a vapor compressor 59. However, it is also possible to feed the vaporous methanol into the reaction column 31 without the use of a vapor compressor 59.

FIG. 3 shows a process flow diagram for a process for preparing alkali metal methoxide in a second embodiment.

The embodiment shown in FIG. 3 differs from the embodiment shown in FIG. 2 in that the vapor compressor 59 with which the proportion of the vaporous methanol which has been withdrawn from the top of the distillation column 1 via the vapor draw 5 and has not been condensed out in the condenser 55 is compressed, is not present.

However, in the embodiment shown in FIG. 3, a further vapor compressor 61 is positioned in the vapor draw 47 from the reaction column 31, which opens into the distillation column 1 as feed 3. In the vapor compressor 61, energy is additionally supplied for the distillation to the methanol/water mixture withdrawn from the reaction column 31 via the vapor draw 47. Firstly, the methanol/water mixture can be compressed to the pressure of the distillation column 1 when the distillation column 1 is operated at a higher pressure than the reaction column 31. Alternatively, it is also possible to compress the methanol/water mixture to a higher pressure than the pressure with which the distillation column 1 is operated, such that the methanol/water mixture is abruptly decompressed on entry into the distillation column 1.

FIG. 4 shows a process flow diagram for a process for preparing alkali metal methoxide in a third embodiment. In contrast to the embodiment shown in FIG. 2, a separate reaction column 31 and a distillation column 1 are not provided, and the columns are instead accommodated in a single column jacket in the form of a dividing wall column 71.

In the dividing wall column 71, the lower region is separated by a dividing wall 73 into a first subregion 75 and a second subregion 77. The first subregion 75 serves as a reaction column and the second subregion 77 as a stripping section of the distillation column.

At the upper end of the first subregion 75, aqueous alkali metal hydroxide solution which has been heated in the heat transferer 35 and has optionally been admixed with methanol or a methanol/water mixture is fed in via the feed 33. In the lower region of the first subregion 75, methanol is fed in vaporous form, via the methanol feed 37. In addition, as likewise shown in FIG. 2, methanol can likewise be fed in vaporous form via further methanol feeds 39.

At the bottom of the first subregion 75, the product, alkali metal methoxide in methanol, is withdrawn via the bottom outlet 41. A portion of the stream withdrawn via the bottom outlet 41 is recycled via the evaporator 43 and the return line 45 into the first subregion 75 of the dividing wall column 71. The return line 45 opens into the first subregion 75 of the dividing wall column 71 preferably below the methanol feed 37. The methanol/water mixture formed in the reaction flows in vaporous form over the upper end of the dividing wall 73 into the upper part 79 of the dividing wall column, which acts as the rectifying section of the distillation column 1. The second subregion 77 of the dividing wall column 71 serves as the stripping section for distillation of the methanol/water mixture. Via the vapor draw 5 on the upper part 79 of the dividing wall column 71, the essentially methanol-comprising vapor is drawn off from the distillation. As shown in FIGS. 2 and 3, a substream of the essentially methanol-comprising vapor stream is fed via the line 51 to the vapor compressor 17, in which the essentially methanol-comprising vapor stream is compressed. The compressed vapor stream then serves as heating vapor in the evaporator 11. In the embodiment shown here, the evaporator 11 is also arranged as an intermediate evaporator in the stripping section of the part of the dividing wall column 71 which serves as the distillation column. The heat transfer to the methanol/water mixture to be distilled condenses a portion of the essentially methanol-comprising vapor stream. The condensed portion is recycled via the feed 21 into the upper part 79, preferably at the top of the dividing wall column 71. Likewise at the top of the dividing wall column, the methanol feed 57, with which fresh methanol can be metered in, opens out, in order to replace the methanol consumed in the reaction to give alkali metal methoxide. The proportion of the essentially methanol-comprising vapor stream which has not been condensed in the evaporator 11 is fed via the vapor line 53 to the condenser 55. In the condenser 55, the proportion of the essentially methanol-comprising vapor stream which has been conducted through the vapor line 53 and the proportion of the essentially methanol-comprising vapor stream which has not been conducted through the vapor compressor 17 are partly condensed. The condensed portion is likewise recycled into the dividing wall column 71 via the top of the dividing wall column 71. The uncondensed portion is recycled into the dividing wall column 71 via the vapor compressor 59 and the methanol feeds 37 and 39 into the first subregion 75, which serves as the reaction column.

The dividing wall column is preferably operated at a pressure in the range from 0.2 to 10 bar, especially at a pressure in the range from 0.5 to 3 bar.

At the bottom of the second subregion 77, an essentially water-comprising bottom stream is withdrawn via the bottom outlet 7. A portion of the essentially water-comprising bottom stream is at least partly evaporated in the evaporator 49 and recycled into the second subregion 77, preferably at the bottom. The second evaporator 49 is heated conventionally, preferably with heating vapor.

The vapor compressor used in all embodiments may, for example, be a one-stage or multistage turbocompressor or a screw compressor. The technical detailed configuration of the vapor compressor 17 is generally guided by the manufacturer's instructions and is familiar to the person skilled in the art. For example, it is possible to connect a droplet separator upstream of the vapor compressor 17.

When an evaporator 11 which serves as an intermediate evaporator is heated with the essentially methanol-comprising vapor stream compressed in the vapor compressor 17, the dimensions of the area of the evaporator 11 are preferably such that the temperature difference is not more than 50 K, especially not more than 30 K. Small temperature differences can reduce the required drive output of the vapor compressor 17. The essentially methanol-comprising vapor stream is compressed in the vapor compressor 17 preferably by a factor in the range from 1.5 to 19, especially in the range from 2 to 4. The exact value of the pressure increase in the vapor compressor 17 is guided by the dimensions of the heat transfer area of the evaporator 11.

The dividing wall 73 can, as shown here, divide the column cross section into two circle segments, but it is alternatively also possible to configure the dividing wall coaxially, in which case it is preferred that the stripping section of the distillation is configured in the inner region and the reaction performed in the annular section surrounding the dividing wall.

In the embodiment shown in FIG. 4 too, it is possible to use the condensed proportion of the essentially methanol-comprising vapor stream which has been withdrawn from the evaporator 11 to further heat the vaporous proportion of the essentially methanol-comprising vapor stream which has been withdrawn from the condenser 55 in a suitable heat transferer.

In the embodiments shown in FIGS. 3 and 4, inerts can be discharged in the same way as in the embodiment shown in FIG. 2. Alternatively, it is also possible to position a condenser for discharge of the inerts downstream of the vapor compressor 11 in the line 53.

### LIST OF REFERENCE NUMERALS

1 Distillation column
3 Feed
5 Vapor draw
7 Bottom outlet
9 Outlet
11 Evaporator
13 Feed
15 Valve
17 Vapor compressor
19 Throttle
21 Feed
23 Methanol draw
25 Valve
31 Reaction column
33 Feed
35 Heat transferer
37 Methanol feed
39 Further methanol feed
41 Bottom outlet
43 Evaporator
45 Return line
47 Vapor draw
49 Evaporator
51 Line
53 Vapor line
55 Condenser
57 Methanol feed
59 Vapor compressor
61 Further vapor compressor
63 Line
65 Condenser
67 Return line
71 Dividing wall column
73 Dividing wall
75 First subregion
77 Second subregion
79 Upper section

1-12. (canceled)

13. A process for distillative workup of a methanol/water mixture, comprising:
(a) adding the methanol/water mixture to a distillation column, wherein the methanol/water mixture is supplied to the distillation column in gaseous form and wherein the methanol/water mixture is withdrawn as a top stream from a reaction column for preparing alkali metal methoxides,
(b) withdrawing an at least 99 wt-% methanol comprising vapor stream at the top of the distillation column and an essentially water-comprising bottom stream at the bottom of the distillation column,
(c) compressing at least a portion of the at least 99 wt-% methanol comprising vapor stream to form a compressed vapor stream,
(d) adding the compressed vapor stream as heating vapor to an evaporator in which at least a portion of the methanol/water mixture to be separated is evaporated.

14. The process according to claim 13, wherein the evaporator is an intermediate evaporator.

15. The process according to claim 14, wherein the distillation column contains a stripping section and a feed point, and wherein the intermediate evaporator is arranged in the stripping section of the distillation column or in the region of the feed point of the methanol/water mixture.

16. The process according to claim 14, wherein the intermediate evaporator is arranged such that the distillation column has 1 to 50 theoretical plates below the intermediate evaporator and 1 to 200 theoretical plates above the intermediate evaporator.

17. The process according to claim 14, wherein the diameter of the distillation column above the intermediate evaporator is greater than below the intermediate evaporator.

18. The process according to claim 13, which further comprises a heated evaporator arranged at the bottom of the distillation column.

19. The process according to claim 13, wherein the distillation column is operated at a pressure in the range from 0.2 to 10 bar.

20. The process according to claim 13, wherein a portion of the at least 99 wt-% methanol comprising vapor stream is condensed and passed back into the distillation column.

21. A process for preparing alkali metal methoxides in a reaction column which comprises adding methanol and alkali metal hydroxide solution to the reaction column, withdrawing alkali metal methoxide dissolved in methanol at the lower end of the reaction column and withdrawing a methanol/water mixture at the upper end of the reaction column, and working up the methanol/water mixture by the process according to claim 13.

22. The process according to claim 21, wherein the reaction column and the distillation column for working up the methanol/water mixture are accommodated in one column jacket, the lower region of the column being divided by a dividing wall.
